(19) United States
(12) Patent Application Publication
(10) Pub. No.: US 2004/0137525 A1
(43) Pub. Date: Jul. 15, 2004

(54) Detection of salmonella cells by fluorescence polarization

(76) Inventors: Michael E. Jolley, Round Lake, IL (US); Mohammad Sarwar Nasir, Grayslake, IL (US)

Correspondence Address: Richard A. Machonkin, McDonnell Boehnen Hulbert & Berghoff, 32nd Floor, 300 S. Wacker Drive, Chicago, IL, 60606, US

(21) Appl. No.: 10/686,053

(22) Filed: Oct. 14, 2003

Related U.S. Application Data
(60) Provisional application No. 60/419,020, filed on Oct. 16, 2002.

(51) Int. Cl. G01N 33/53

(52) U.S. Cl. 435/7.1

(57) ABSTRACT

A homogeneous fluorescence polarization inhibition assay is used to test for Salmonella contamination, e.g., Salmonella cells, in a sample. The assay makes use of a tracer comprising a fluorophore conjugated to an oligosaccharide from a Salmonella cell wall lipopolysaccharide. The sample is added to an anti-Salmonella antibody to form a mixture, and a blank fluorescence polarization measurement is taken. The tracer is then added to the mixture. After incubation, the fluorescence polarization of the mixture is measured and the blank reading is subtracted. The level of Salmonella contamination in the sample may be determined from the fluorescence polarization measured in this way.

### CROSS-REFERENCE TO RELATED APPLICATIONS

This application claims the benefit of U.S. Provisional Application No. 60/419,020, filed on Oct. 16, 2002, which is incorporated herein by reference.

### BACKGROUND OF THE INVENTION

1. Field of the Invention

This invention relates to the field of diagnostic assays. More particularly, this invention relates to an inhibition assay that uses fluorescence polarization for the detection of Salmonella cells.

2. Description of Related Art

Fluorescence polarization is a useful technology for the detection of the binding of small molecules to larger ones. The underlying principle of the technology is that fluorescence polarization can serve as a measure of the rate of rotation of fluorescent molecules and, hence, their size. In particular, small molecules have a fast rotation rate and therefore tend to have a low fluorescence polarization, whereas large molecules rotate more slowly and therefore tend to have a higher fluorescence polarization. The principles and applications of fluorescence polarization have been described in a recent review. See M. S. Nasir and M. E. Jolley, Combinatorial Chemistry and High Throughput Screening, 1999, Z 177-190.

Fluorescence polarization assays have been developed for the detection of serum antibodies to Brucella abortus (See K. Nielsen, et al., J. Immunol. Methods, 1996, 195, 161-168), Mycobacterium bovis (See M. Lin, et al., Clin. Diagnos. Lab. Immun., 1996, 3, 438-443), equine infectious anemia virus (See S. B. Tencza, et al., J. Clin. Microbiol., 2000, 38, 1854-1859), and Salmonella enteriditis (See M. S. Nasir, et al., Proceedings of the 104^{th} Annual Meeting of the United States Animal Health Association, 2000, 527-535).

However, in order to screen for Salmonella contamination in food products, it is desirable to detect Salmonella cells directly. Currently, this is often done by first culturing a sample, such as on an agar plate. The culture is then tested for Salmonella by exposure to antiserum in an agglutination assay. Such agglutination assays, however, are not quantitative (indeed, they are potentially subjective) and often suffer from low sensitivity. Accordingly, there is a need for objective and sensitive assays specific for Salmonella cells that are rapid and easy to perform.

### SUMMARY OF THE INVENTION

In a first principal aspect, the present invention provides a method for detecting Salmonella antigens in a sample. In accordance with the method, the sample is combined with a tracer and an anti-Salmonella antibody to form an assay mixture. The tracer comprises a fluorophore conjugated to an oligosaccharide from a Salmonella cell wall lipopolysaccharide. The tracer is able to bind to the anti-Salmonella antibody to produce a detectable change in fluorescence polarization. The fluorescence polarization of the assay mixture is measure to obtain a measured fluorescence polarization value. The measured fluorescence polarization value is related to the concentration of Salmonella antigens in the sample.

In a second principal aspect, the present invention provides a method for testing for Salmonella contamination. In accordance with the method, a sample containing Salmonella cells is obtained. The sample is cultured in a culture medium to provide a cultured sample. The cultured sample is then autoclaved to provide an autoclaved sample. The autoclaved sample is combined with anti-Salmonella antibody to provide a first mixture. The fluorescence polarization of the first mixture is measured to obtain a first fluorescence polarization value. The first mixture is combined with a tracer to provide a second mixture. The tracer comprises a fluorophore conjugated to an oligosaccharide from a Salmonella cell wall lipopolysaccharide. The tracer is able to bind to the anti-Salmonella antibody to produce a detectable change in fluorescence polarization. The second mixture is incubated for a predetermined period of time. The fluorescence polarization of the mixture is measured to obtain a second fluorescence polarization value. The first fluorescence polarization value is subtracted from the second fluorescence polarization value to obtain a corrected fluorescence polarization value. The corrected fluorescence polarization value is related to the level of Salmonella contamination in the sample.

In a third principal aspect, the present provides an assay kit for testing for Salmonella contamination in a sample. The assay kit comprises an anti-Salmonella antibody and a tracer, each in an amount suitable for at least fluorescence polarization assay to test for Salmonella concentration in the sample, packaging, and instructions for using the anti-Salmonella antibody and tracer in the fluorescence polarization assay. The tracer comprises a fluorophore conjugated to an oligosaccharide from a Salmonella cell wall lipopolysaccharide. The tracer is able to bind to the anti-Salmonella antibody to produce a detectable change in fluorescence polarization.

### BRIEF DESCRIPTION OF THE DRAWINGS

FIG. 1 is a plot showing the measured fluorescence polarization of the SE.1, SE.5, ST.01, and ST.1 tracers after varying amounts of the AB1 antiserum were added.

FIG. 2 is a plot showing the measured fluorescence polarization of the SE.1 tracer after varying amounts of the AB2 antiserum were added and of the ST.01 tracer after varying amounts of the AB3 antiserum were added.

FIG. 3 is a plot showing the measured fluorescence polarization for samples containing varying levels of Salmonella enteriditis cells (using the AB1 antiserum and the SE.1 and SE.5 tracers) and for samples containing varying levels of Salmonella typhimurium cells (using the AB1 antiserum and the ST.01 and ST.1 tracers).

FIG. 4 is a plot showing the measured fluorescence polarization for samples containing varying levels of Salmonella enteriditis cells (using the AB2 antiserum and the SE.1 and SE.5 tracers) and for samples containing varying levels of Salmonella typhimurium cells (using the AB3 antiserum and the ST.01 and ST.1 tracers).

FIG. 5 is a plot showing the measured fluorescence polarization of Salmonella newport and Salmonella montevideo cultured on agar plates.

FIG. 6 is a plot showing the measured fluorescence polarization of Salmonella newport and Salmonella montevideo cultured on agar plates.

### DETAILED DESCRIPTION OF THE PREFERRED EMBODIMENTS

The present invention provides a homogeneous fluorescence polarization inhibition assay for detection of Salmonella cells in samples, such as samples from food products or animal feces. The sample may be cultured in a culture medium, such as a broth or an agar plate, before the assay is performed. The assay makes use of a tracer comprising a fluorophore conjugated to an oligosaccharide from a Salmonella cell wall lipopolysaccharide. A sample suspected of containing Salmonella cells is added to an anti-Salmonella antibody to form a mixture, and a blank fluorescence polarization measurement is taken. The tracer is then added to the mixture. After incubation, the fluorescence polarization of the mixture is measured and the blank reading is subtracted. The level of Salmonella contamination in the sample may be determined from the fluorescence polarization measured in this way.

This fluorescence polarization approach has been found to be very sensitive, specific, rapid, quantitative, inexpensive, and robust. Moreover, in exemplary embodiments, the tracers used in this inhibition assay to detect Salmonella cells are the same as may be used to detect serum antibodies to Salmonella. Accordingly, Salmonella detection kits may be used both for screening for Salmonella infection in live animals, by detecting serum antibodies, and for testing for the presence Salmonella cells in food products and animal feces.

### EXAMPLE 1

### FPIAs for Broth-Cultured Salmonella entiriditis and Salmonella typhimurium

Fluorescence polarization inhibition assays (FPIAs) for the detection of Salmonella enteriditis (SE) and Salmonella typhimurium (ST) cells have been developed using three antisera and four fluorescently-labeled preparations (tracers) of the O-polysaccharides (OPSs) from SE (SE.1 and SE.5) and ST (ST.01 and ST.1). A bovine anti-ST antiserum (AB1) recognized all tracers. A specific rabbit anti-Salmonella Group D1 antiserum (AB2) recognized the SE tracers but did not react with the ST tracers. Conversely, a specific rabbit anti-Salmonella Group B antiserum (AB3) recognized the ST tracers but not the SE tracers. None of the tracers recognized rabbit specific anti-E. coli 0157 or anti-Salmonella Group E antisera (all O-antigens). Chicken specific anti-Salmonella pullorum (SP) antisera reacted strongly to these tracers.

Cells were grown in culture overnight and autoclaved (15 minutes at 121° C.). 20 μl of autoclaved sample was added to one ml of an appropriately diluted antiserum in buffer and a fluorescence blank obtained (Sentry-FP™ instrument; Diachemix Corp.). To this mixture was added 10 μl of appropriately diluted tracer. The reaction mixture was incubated at room temperature for 4 minutes, and the blank-subtracted fluorescence polarization (FP) of the tracer determined. A sample having a fluorescence polarization less than 10 mP of that of medium alone (5 standard deviations: SDs) was considered positive.

None of the assays cross-reacted with Salmonella montevideo (SM; Group C1) or Escherichia coli (EC; two strains). All assays had a dynamic range of less than 10⁵ to approximately 10⁷ CFUs. Assays that used the AB2 and AB3 antisera were totally specific to SE and to ST, respectively. When the AB1 antiserum was used, the ST tracers were approximately 10% cross-reactive with SE, and the SE tracers were approximately 0.5% cross-reactive with ST.

### 1. Materials and Methods

The lipopolysaccharides (LPSS) from SE and ST were obtained from Sigma Chemical Co., St. Louis, Mo. (catalogue numbers L 6011 and L 6511 respectively). Fluorescein isothiocyanate, isomer I (FITC-I) and media were also obtained from Sigma (catalogue number F 7250). EC strains were obtained from Becton Dickinson Microbiology Systems, Cockeysville, Md. They were catalogue numbers 4337015 and 4337071 (ATCC numbers 25922 and 35218 respectively; EC#1 and EC#2). SE, ST, and SM strains used in the FPIAs were field isolates from chickens.

Bovine anti-ST (Poly Serum®) was provided by Grand Laboratories, Larchwood, Iowa. Rabbit specific anti-Salmonella D1 (O:1,9,12), B (O:1,4,5,12) and E (O:1,3,10,15,19,34) were obtained from Difco Laboratories, Detroit, Mich. (cat. nos. 229511, 22481 and 228191 respectively). Rabbit specific anti-E. coli 0157 was purchased from BBL, Sparks, Md. (cat. no. 0263950). Chicken specific anti-SP (high and low titer and negative) were obtained from NVSL, Ames, Iowa.

The preparation of the tracer, i.e., fluorescently-labeled O-polysaccharides (OPSs) is described in M. S. Nasir et al., Proceedings of the 104^{th} Annual Meeting of the United States Animal Health Association, 2000, 527-535, which is incorporated herein by reference. Briefly, the OPSs were obtained by hydrolysis of the corresponding LPSs by hydrolysis in 1% acetic acid in a boiling water bath and purification by passage through a polymyxin B-agarose column. Fluorescein was attached by alkalization and reaction with FITC-I, purification by Sephadex G25 gel chromatography and A25 anion exchange chromatography. Tracers SE.1 and SE.5 were eluted with 0.1 M sodium phosphate buffer, pH 7.5, and 0.5 M sodium chloride, in the same buffer, respectively. Their yields were approximately equal. Tracers ST.01 and ST.1 were eluted with 0.01 M and 0.1 M sodium phosphate, pH 7.5, respectively. Again, the yields were approximately equal.

SE, ST, and SM cells were grown in tryptic soy broth overnight at 37° C. EC cells were similarly grown in EC medium. Cultures were killed by autoclaving at 121° C. for 15 minutes before assaying. 20 μl of the autoclaved culture was added to 1 ml of diluted antiserum in PBSA (0.01 M sodium phosphate, pH 7.5, containing 9 g/l sodium chloride and 0.1% sodium azide). A fluorescence blank was taken (Sentry-FP™, Diachemix Corp., Grayslake, Ill.) and 10 μl of tracer, diluted in PBSA-BGG (PBSA containing 100 μg/ml bovine gamma globulin) such that a 1:100 dilution gave approximately 1 nM fluorescein equivalents, was then added. The blank-subtracted fluorescence polarization of the tracer was then determined after four minutes.

### 2. Results

The reactions of antisera with tracers SE.1, SE.5, ST.01, and ST.1 are summarized below in Table 1. Unless otherwise stated, the antisera were employed at a dilution of 1:50. It was found that AB2 reacted only with SE tracers and AB3 reacted only with ST tracers, as indicated by the delta mP columns in Table 1. Surprisingly, however, AB1, which was raised against ST, reacted significantly better with the SE tracers than with the ST tracers. Chicken anti-SP antisera reacted well with both SE.5 and ST.1 (SE.1 and ST.01 were not tested), showing the immunological similarity of SE and SP. Rabbit anti-Group E and anti-EC 0157 showed no significant reaction with either of the SE or ST tracers that were tested.

TABLE 1
Assessment of the binding of the tracers with
various antisera (NT = not tested).

| | SE.1 | Delta | SE.5 | Delta | ST.01 | Delta | ST.1 | Delta |
|---|---|---|---|---|---|---|---|---|
| | (mP) | mP | (mP) | mP | (mP) | mP | (mP) | mP |
| Buffer | 93 | | 97 | | 96 | | 93 | |
| AB1 (1/100) | 245 | 153 | 246 | 149 | 180 | 84 | 186 | 93 |
| Buffer | 88 | | 96 | | 99 | | 90 | |
| AB2 (1/50) | 170 | 82 | 181 | 85 | 96 | −3 | 88 | −2 |
| AB3 (1/50) | 88 | 0 | 95 | −2 | 141 | 42 | 141 | 51 |
| O157 (1/50) | 92 | 4 | 100 | 4 | 99 | 0 | 90 | 0 |
| Buffer | NT | | 105 | | NT | | 98 | |
| Anti-SP − | NT | | 114 | 9 | NT | | 98 | 0 |
| Anti-SP + | NT | | 177 | 72 | NT | | 144 | 46 |
| Anti-SP ++ | NT | | 285 | 180 | NT | | 240 | 142 |
| Anti Gp E | NT | | 108 | 3 | NT | | 94 | −4 |

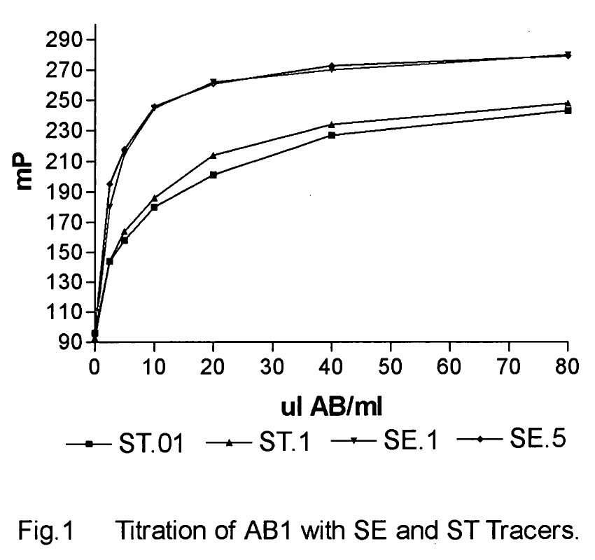

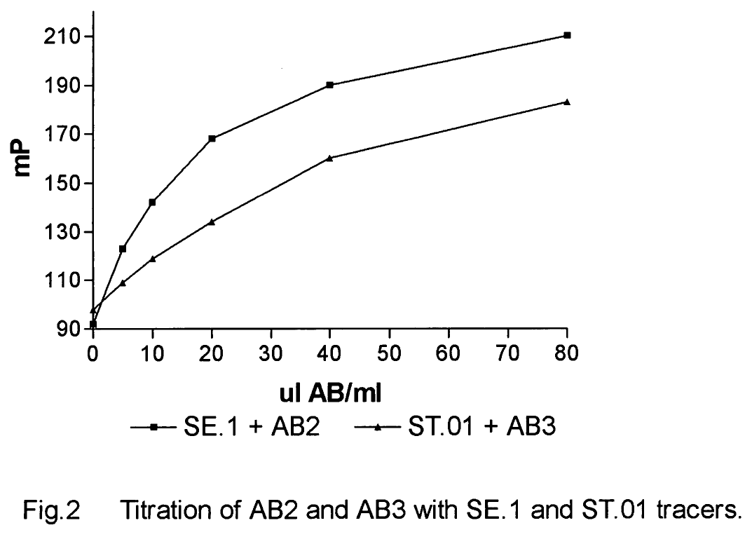

FIG. 1 shows the fluorescence polarization (in mP) as the tracers ST.01, ST.1, SE.1 and SE.5 were titrated with AB1. FIG. 2 shows the fluorescence polarization as SE.1 was titrated with AB2 and ST.01 was titrated with AB3. Surprisingly, SE.1 and SE.5 were equivalent with AB1, as were ST.01 and ST.1. Previous studies have found that the low salt tracers are significantly inferior to the high salt tracers in the detection of chicken antibodies. For this reason, only SE.1 and ST.01 were used for the AB2 and AB3 titrations.

Table 2 below shows the results of the SE.1 and SE.5 tracers, with antisera AB2 and AB1, in FPIAs for ST, SM, EC (two strains), and various levels of SE. Table 3 below shows the results for the ST.01 and ST.1 tracers, with antisera AB3 and AB1, in FPIAs for SE, SM, EC (two strains), and various levels of ST.

TABLE 2
Results of FPIAs using tracers SE.1 and SE.5 and
antisera AB2 and AB1 (ND = not determined).

| | |
|---|---|
| AB2 Rabbit | AB1 Bovine |
| anti-GP D1 (1:50) | anti-ST (1:300) |

| | CFUs | SE.1 | SE.5 | SE.1 | SE.5 |
|---|---|---|---|---|---|
| Sample | (× 10⁻⁵) | (mP) | (mP) | (mP) | (mP) |
| ST | 200 | 176 | 190 | 156 | 161 |
| SM | 200 | 174 | 184 | 172 | 178 |
| EC #1 | >>200 | ND | ND | 175 | ND |
| EC #2 | >>200 | ND | ND | 162 | 175 |
| SE | 200 | 110 | 119 | 101 | 107 |
| SE | 100 | 116 | 130 | 103 | 110 |
| SE | 50 | 126 | 140 | 103 | 115 |
| SE | 25 | 138 | 150 | 111 | 122 |
| SE | 12 | 149 | 161 | 118 | 126 |
| SE | 6 | 160 | 168 | 127 | 137 |
| SE | 3 | 165 | 172 | 141 | 146 |
| SE | 1.5 | 167 | 175 | 152 | 158 |
| SE | 0.8 | 170 | 179 | 166 | 167 |
| SE | 0.4 | 172 | 180 | 172 | 172 |
| Medium | 0 | 177 | 185 | 173 | 179 |

TABLE 3
Results of FPIAs using tracers ST.01 and ST.1 and
antisera AB3 and AB1 (ND = not determined).

| | |
|---|---|
| AB3 Rabbit | AB1 Bovine |
| anti-GP B (1:25) | anti-ST (1:100) |

| | CFUs | ST.01 | ST.1 | ST.01 | ST.1 |
|---|---|---|---|---|---|
| Sample | (× 10⁻⁵) | (mP) | (mP) | (mP) | (mP) |
| SE | 200 | 166 | 171 | 122 | 128 |
| SM | 200 | 166 | 172 | 159 | 172 |
| EC #1 | >>200 | ND) | ND | 160 | 175 |
| EC #2 | >>200 | ND | ND | 162 | 175 |
| ST | 200 | 120 | 111 | 107 | 103 |
| ST | 100 | 127 | 116 | 109 | 108 |
| ST | 50 | 133 | 125 | 113 | 114 |
| ST | 25 | 141 | 134 | 118 | 122 |
| ST | 12 | 147 | 143 | 127 | 132 |
| ST | 6 | 149 | 150 | 136 | 144 |
| ST | 3 | 156 | 158 | 147 | 154 |
| ST | 1.5 | 159 | 161 | 152 | 162 |
| Medium | 0 | 166 | 173 | 162 | 173 |

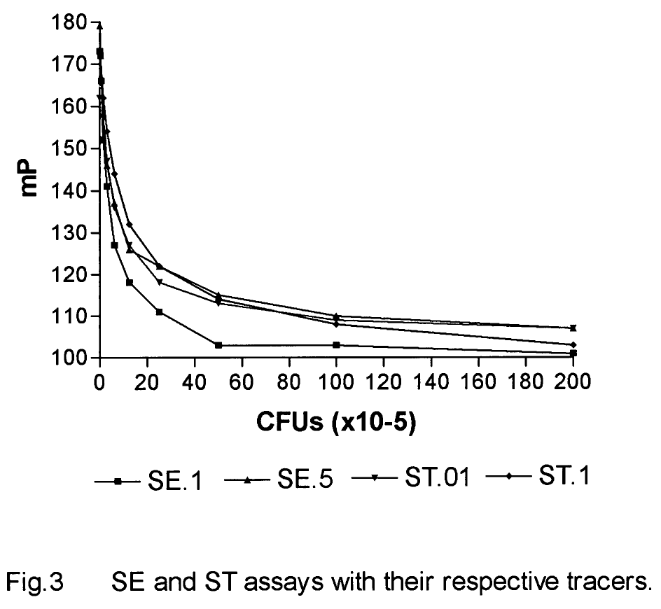

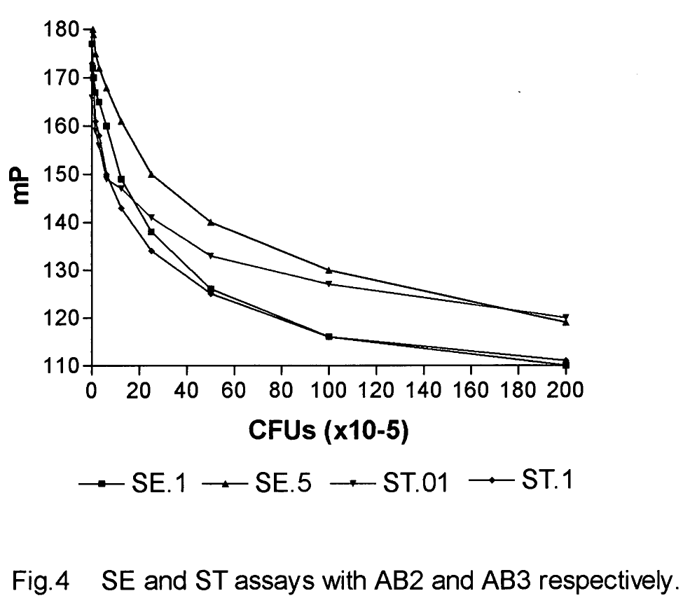

FIG. 3 is a plot of the results of the FPIAs of SE, using antiserum AB1 and tracers SE.1 and SE.5, and of ST, using antiserum AB1 and tracers ST.01 and ST.1. FIG. 4 is a plot of the results of the FPIAs of SE, using antiserum AB2 and tracers SE.1 and SE.5, and of ST, using antiserum AB3 and tracers ST.01 and ST.1. The standard deviations of the media in these assays were approximately 2 mP (data not shown). Therefore, a mP of medium alone minus 10 mP was chosen as the cutoff (99.9% confidence level). The assays had sensitivities of 10⁻⁵ CFUs, or less, and dynamic ranges of approximately two orders of magnitude. The assays using the SE tracers and the AB1 antisterum had a cross-reactivity with ST of approximately 0.5%, and the assays using the ST tracers and the AB1 antiserum had a cross-reactivity with SE of approximately 10%. The assays that used the AB2 and AB3 antisera were totally specific to SE and to ST, respectively. SM and EC (two strains) exhibited no cross-reactivity in any of the assays.

### 3. Conclusions

It has been found that Salmonella enteriditis (SE) and Salmonella typhimurium (ST) cells may be detected in fluorescence polarization inhibition assays (FPIAs) employing fluorescently-labeled O-polysaccharides (OPSs) from the corresponding bacterial lipopolysaccharides (LPSs) as tracers and appropriate antisera. The FPIAs are very rapid, sensitive, and simple to perform. They may also be quantitative and made to be field portable. Furthermore, the reagents used in the FPIAs are very stable and low cost.

### EXAMPLE 2

### FPIAs for Detecting Salmonella Cells in Colonies Cultured on Agar Plates

The FPIA approach has also been shown to be able to detect Salmonella cells in colonies cultured on agar plates. The overall approach is as follows. Samples suspected of containing Salmonella cells are cultured on agar plates until visible colonies develop. A colony is then removed from the plate, suspended in 1 ml of water and boiled for five minutes. 100 μl is then added to 1 ml of a solution containing antibodies specific to the Salmonella species being tested (e.g., an appropriate antiserum diluted in buffer), and the mixture is mixed well. A blank fluorescence polarization measurement is taken of the mixture and then 10 μl of tracer is added. The resulting mixture is then incubated for 2 minutes, and its fluorescence polarization is measured, subtracting the blank measurement.

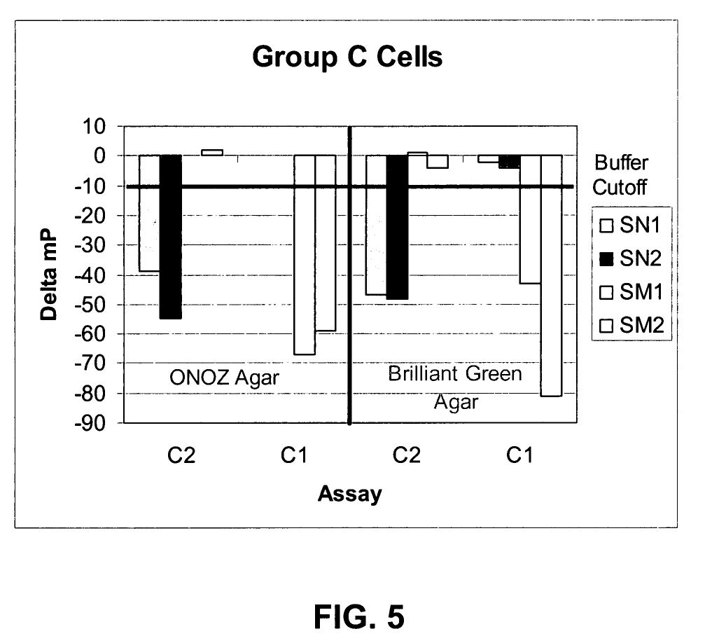

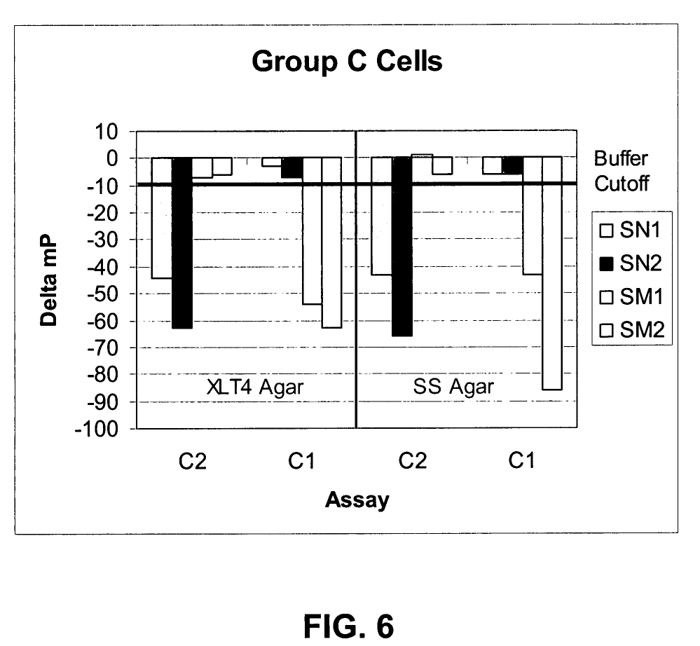

Table 4 below shows the results for FPIA assays conducted on Salmonella newport (SN1 and SN2) and Salmonella montevideo (SM1 and SM2) cultured on “ONOZ” and “Brilliant Green” agar plates. These assays were performed using tracers C1 and C2 prepared from the OPSs of Salmonella montevideo and Salmonella newport, respectively, in a manner similar to that described above with respect to Salmonella enteriditis and Salmonella typhimurium, and the corresponding antisera. FIG. 5 graphically illustrates the results of Table 4. Table 5 below shows similar FPIA results, except that the Salmonella montevideo and Salmonella newport were cultured on “XLT4” and on “SS” agar plates. FIG. 6 graphically illustrates the results of Table 5.

TABLE 4
FPIA results for Salmonella newport and
Salmonella montevideo cultured on agar plates.

| | ONOZ Agar | | | | Brilliant Green Agar | | | |
|---|---|---|---|---|---|---|---|---|
| | C2 | | C1 | | C2 | | | |
| | tracer | Delta | tracer | Delta | tracer | Delta | C1 tracer | Delta |
| | (mP) | mP | (mP) | mP | (mP) | mP | (mP) | mP |
| PBSA | 181 | | 180 | | 186 | | 186 | |
| SN1 | 142 | −39 | 180 | 0 | 139 | −47 | 184 | −2 |
| SN2 | 126 | −55 | 180 | 0 | 138 | −48 | 182 | −4 |
| SM1 | 181 | 0 | 113 | −67 | 187 | 1 | 133 | −43 |
| SM2 | 183 | 2 | 121 | −59 | 182 | −4 | 105 | −81 |

TABLE 5
FPIA results for Salmonella newport and
Salmonella montevideo cultured on agar plates.

| | XLT4 Agar | | | | SS Agar | | | |
|---|---|---|---|---|---|---|---|---|
| | C2 | | C1 | | C2 | | | |
| | tracer | Delta | tracer | Delta | tracer | Delta | C1 tracer | Delta |
| | (mP) | mP | (mP) | mP | (mP) | mP | (mP) | mP |
| PBSA | 184 | | 190 | | 184 | | 190 | |
| SN1 | 140 | −44 | 187 | −3 | 141 | −43 | 184 | −6 |
| SM1 | 177 | −7 | 136 | −54 | 187 | 1 | 133 | −43 |
| PBSA | 178 | | 184 | | 178 | | 184 | |
| SN2 | 115 | −63 | 177 | −7 | 112 | −66 | 178 | −6 |
| SM2 | 172 | −6 | 121 | −63 | 172 | −6 | 98 | −86 |

FPIA assays were also performed on Salmonella typhi (STy21a) samples cultured on “Brilliant Green” agar plates. These FPIAs assays used the Salmonella enteriditis tracer SE.5 and the Salmonella typhimurium tracer ST.1 described above, along with corresponding antisera. The results are summarized in Table 6 below.

TABLE 6
FPIA results for Salmonella typhi cultured on agar.

| | SE.5 tracer | | ST.1 tracer | |
|---|---|---|---|---|
| | (mP) | Delta mP | (mP) | Delta mP |
| PBSA | 176 | | 168 | |
| STy21a-2-1 | 145 | −31 | 169 | 1 |
| STy21a-2-2 | 130 | −46 | 169 | 1 |

These results show that the FPIA approach may be used to detect many different Salmonella species, whether they are cultured in broth or on agar plates.

### EXAMPLE 3

### Assay Kit

The materials used to perform the assay of the present invention may be made available in kit form. The kit preferably includes tracer and anti-Salmonella antibody in an amount suitable for at least one assay, along with suitable packaging and instructions for using the tracer and antibody to test for Salmonella contamination in a sample. The sample could be from a food product or from animal feces, for example. The sample could also be cultured in a culture medium, such as an agar plate or a broth, before the assay is performed on it.

The tracer and antibody may be provided in solution, as a liquid dispersion, or as a substantially dry powder (e.g., in lyophilized form). The suitable packaging can be any solid matrix or material, such as glass, plastic, paper, foil, and the like, capable of separately holding within fixed limits the buffer, tracer, and antibody. For example the tracer and antibody may be provided as solutions in separate labeled bottles or vials made of glass or plastic. The tracer comprises a fluorophore, such as fluorescein isothiocyanate, isomer I, conjugated to an oligosaccharide from a Salmonella cell wall lipopolysaccharide. Other fluorophores may be used, provided the resulting tracer is able to bind with the anti-Salmonella antibody to produce a detectable change in fluorescence polarization.

The foregoing description of the invention is presented for purposes of illustration and description, and is not intended, nor should be construed, to be exhaustive or to limit the invention to the precise forms disclosed. The description was selected to best explain the principles of the invention and practical application of these principles to enable others skilled in the art to best utilize the invention in various embodiments and with various modifications as are suited to the particular use contemplated. It is intended that the scope of the invention not be limited by the specification, but defined by the claims.

What is claimed is:

1. A method for detecting Salmonella antigens in a sample, said method comprising the steps of:
combining said sample with a tracer and an anti-Salmonella antibody to form an assay mixture, said tracer comprising a fluorophore conjugated to an oligosaccharide from a Salmonella cell wall lipopolysaccharide, said tracer being able to bind to said anti-Salmonella antibody to produce a detectable change in fluorescence polarization; and
measuring the fluorescence polarization of said assay mixture to obtain a measured fluorescence polarization value, wherein said measured fluorescence polarization value is related to the concentration of Salmonella antigens in said sample.

2. The method of claim 1, wherein said fluorophore is fluorescein isothiocyanate, isomer I.

3. The method of claim 1, wherein said sample is a cultured sample.

4. The method of claim 1, wherein said sample is from a food product.

5. The method of claim 1, wherein said sample is from animal feces.

6. The method of claim 1, wherein combining said sample with a tracer and an anti-Salmonella antibody to form an assay mixture comprises:
combining said sample with said anti-Salmonella antibody to provide a blank mixture; and
combining said blank mixture with said tracer to provide said assay mixture.

7. The method of claim 6, further comprising:
measuring the fluorescence polarization of said blank mixture to provide a blank fluorescence polarization value.

8. The method of claim 7, further comprising:
subtracting said blank polarization value from said measured fluorescence polarization value to provide a blank-corrected fluorescence polarization value, wherein said measured fluorescence polarization value is related to the concentration of Salmonella antigens in said sample.

9. A method for testing for Salmonella contamination, said method comprising the steps of:
obtaining a sample containing Salmonella cells;
culturing said sample in a culture medium to provide a cultured sample;
autoclaving said cultured sample to provide an autoclaved sample;
combining said autoclaved sample with an anti-Salmonella antibody to provide a first mixture;
measuring the fluorescence polarization of said first mixture to obtain a first fluorescence polarization value;
combining said first mixture with a tracer to provide a second mixture, said tracer comprising a fluorophore conjugated to an oligosaccharide from a Salmonella cell wall lipopolysaccharide, said tracer being able to bind to said anti-Salmonella antibody to produce a detectable change in fluorescence polarization;
incubating said second mixture for a predetermined period of time;
measuring the fluorescence polarization of said second mixture to obtain a second fluorescence polarization value; and
subtracting said first fluorescence polarization value from said second fluorescence polarization value to obtain a corrected fluorescence polarization value, wherein said corrected fluorescence polarization value is related to the level of Salmonella contamination in said sample.

10. The method of claim 9, wherein said fluorophore is fluorescein isothiocyanate, isomer I.

11. The method of claim 9, wherein said sample is from a food product.

12. The method of claim 9, wherein said sample is from animal feces.

13. The method of claim 9, wherein said predetermined of period of time is at least four minutes.

14. An assay kit for testing for Salmonella contamination in a sample, said assay kit comprising:
an anti-Salmonella antibody and a tracer, each in an amount suitable for at least one fluorescence polarization assay to test for Salmonella contamination in said sample, packaging, and instructions for using said anti-Salmonella antibody and said tracer in said fluorescence polarization assay, said tracer comprising a fluorophore conjugated to an oligosaccharide from a Salmonella cell wall lipopolysaccharide, said tracer being able to bind to said anti-Salmonella antibody to produce a detectable change in fluorescence polarization.

15. The assay kit of claim 14, wherein said fluorophore is fluorescein isothiocyanate, isomer I.

16. The assay kit of claim 14, wherein said sample is a cultured sample.

17. The assay kit of claim 14, wherein said sample is from a food product.

18. The assay kit of claim 14, wherein said sample is from animal feces.
